(19) United States
(12) Patent Application Publication
(10) Pub. No.: US 2011/0046361 A1
(43) Pub. Date: Feb. 24, 2011

(54) METHOD FOR SEPARATION OF DOUBLE-STRANDED AND SINGLE-STRANDED NUCLEIC ACIDS

(75) Inventor: Miao Jiang, Cedar Knolls, NJ (US)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE Healthcare Bio-Sciences Corp., Piscataway, NJ (US)

(21) Appl. No.: 12/990,242

(22) PCT Filed: Apr. 22, 2009
(86) PCT No.: PCT/US09/41342

§ 371 Date: Oct. 29, 2010

Related U.S. Application Data
(60) Provisional application No. 61/048,979, filed on Apr. 30, 2008.

(51) Int. Cl. C07H 1/06

(52) U.S. Cl. 536/25.41

(57) ABSTRACT

The invention provides systems, methods and kits for the separation and/or purification of double-stranded and single-stranded nucleic acids. The method includes first mixing a sample containing the double-stranded nucleic acid and the single-stranded nucleic acid with a pH-neutral, buffered solution consisting essentially of a chaotropic salt and a pH buffer to generate a mixture; then applying the mixture to a first mineral support for the double-stranded nucleic acid to bind; and collecting the flow-through which contains unbound single-stranded nucleic acid. The method further includes adjusting the pH of the flow-through to an acidic pH, and applying the acidified flow-through to a second mineral support for the single-stranded nucleic acid to bind. Alternatively the flow-through can be mixed with a lower aliphatic alcohol prior to loading of the second column. The double-stranded and the single-stranded nucleic acids bound can be eluted from the mineral supports respectively.

### CROSS-REFERENCE TO RELATED APPLICATION

This application is a filing under 35 U.S.C. §371 and claims priority to international patent application number PCT/US2009/041342 filed Apr. 22, 2009, published on Nov. 5, 2009 as WO 2009/134652, and claims priority to U.S. provisional patent application No. 61/048,979 filed Apr. 30, 2008; the disclosure of which is incorporated herein by reference in its entirety.

### FIELD OF THE INVENTION

This invention relates to methods for the separation of double-stranded and single-stranded nucleic acids. More specifically, it relates to a simple and rapid system and method for the separation and purification of double-stranded nucleic acid such as DNA from single-stranded nucleic acid such as RNA.

### BACKGROUND OF THE INVENTION

The last three decades has seen considerable effort in the development of improved methods for the isolation and purification of nucleic acids. This has been due mainly to the increasing applications of nucleic acids in the medical and biological sciences. Genomic DNA isolated from blood, tissue or cultured cells has several applications, which include PCR, sequencing, genotyping, hybridization and southern blotting. Plasmid DNA has been utilized in sequencing, PCR, in the development of vaccines and in gene therapy. Isolated RNA has a variety of downstream applications, including in vitro translation, cDNA synthesis, RT-PCR and for microarray gene expression analysis.

The analysis and in vitro manipulation of nucleic acids is typically preceded by an isolation step in order to free the samples from unwanted contaminants which may interfere with subsequent processing procedures. For the vast majority of procedures in both research and diagnostic molecular biology, extracted nucleic acids are required as the first step.

The increased use of DNA and RNA has created a need for fast, simple and reliable methods and reagents for isolating DNA and RNA. In many applications, collecting the biological material sample and subsequent analysis thereof would be substantially simplified if they could be simultaneously isolated from a single sample. The simultaneous isolation is especially important when the sample size is so small, such as in biopsy, that it precludes its separation into smaller samples to perform separate isolation protocols for DNA and RNA.

Also needed is an improved process for separating double-stranded from single-stranded nucleic acids in general. Many nucleic acid molecular biology experiments start from purified nucleic acids and produce a mixture containing both. A separation step is required at the end of many of these experiments and sometimes both the single-stranded and the double-stranded nucleic acids need to be recovered for further analysis.

Currently, the silica membrane column format is widely used for separating and isolating double-stranded and single-stranded nucleic acids. However alcohol is required as a binding reagent, which posts a safety concern as it is flammable.

A novel and advantageous method for carrying out separation and isolation of double-stranded and single-stranded nucleic acids from the same sample is presented herein.

### SUMMARY OF THE INVENTION

In general, the instant invention provides improved methods, systems and kits for rapid separation and isolation of double-stranded and single-stranded nucleic acids from the same sample. The double-stranded nucleic acid is selectively adsorbed to a mineral support in the presence of high concentration of chaotropic salt. The flow-through containing single-stranded nucleic acid is adjusted so that single-stranded nucleic acid is adsorbed to a second mineral support. The nucleic acids are then eluted from each of the mineral supports respectively.

Thus, one aspect of the invention provides a method for the separation and/or purification of double-stranded nucleic acid and single-stranded nucleic acid from a sample. The method includes first mixing a sample containing both the double-stranded nucleic acid and the single-stranded nucleic acid with a pH-neutral, buffered solution consisting essentially of a chaotropic salt and a pH buffer to generate a mixture; then applying the mixture to a first mineral support for double-stranded nucleic acid to bind; and collecting the flow-through which contains unbound single-stranded nucleic acid. The method further includes adjusting the pH of the flow-through to an acidic pH; then loading acidified flow-through to a second mineral support for the single-stranded nucleic acid to bind. Alternatively the flow-through can be mixed with a lower aliphatic alcohol followed by loading to and purification from a second column.

In certain embodiments, the method further comprises eluting the double-stranded nucleic acid from the first mineral support after an optional wash step. In other embodiments, the method also includes eluting the single-stranded nucleic acid from the second mineral support after an optional wash step

In another aspect, the invention provides a kit for separating and isolating double-stranded nucleic acid and single-stranded nucleic acid. The kit includes a pH-neutral, buffered solution consisting essentially of a chaotropic salt and a pH buffer for sample processing; a first mineral support for binding the double-stranded nucleic acid; a second mineral support for binding the single-stranded nucleic acid; an optional elution solution for eluting the double-stranded nucleic acid from the first mineral support; and an optional elution solution for eluting single-stranded nucleic acid from the second mineral support. Optionally, the kit also includes washing solutions for washing the respective mineral supports prior to elution.

In a preferred embodiment, the first mineral support and the second mineral support are each silica membranes.

The above and further features and advantages of the instant invention will become clearer from the following detailed description and claims.

### BRIEF DESCRIPTION OF THE DRAWINGS

FIG. 1 presents a schematic diagram of the method for the separation and purification of double-stranded and single-stranded nucleic acids from a sample, according to an embodiment of the invention.

FIG. 2 shows gel images of genomic DNA and total RNA separation according to an embodiment of the invention. Left side panel shows genomic DNA eluted from the first column. Right side panel shows total RNA eluted from the second column.

### DETAILED DESCRIPTION OF THE INVENTION

The present invention provides compositions, methods, and kits for highly effective, simple separation of double-stranded and single-stranded nucleic acids from the same sample. The nucleic acids can be isolated utilizing the methods of the invention in as little as 10 minutes. The resulting double-stranded and single-stranded nucleic acids are of high quality suitable for use in downstream applications.

The invention is well suited for the separation of double-stranded and single-stranded nucleic acids from a broad range of samples. These include a variety of biological sources such as, without limitation, whole tissues, including biopsy materials and aspirates; in vitro cultured cells, including primary and secondary cells, transformed cell lines, and tissue and blood cells; and body fluids such as urine, sputum, semen, secretions, eye washes and aspirates, lung washes and aspirates. Fungal and plant tissues, such as leaves, roots, stems, and caps, are also within the scope of the present invention. Microorganisms and viruses that may be present on or in a biological sample are within the scope of the invention. Bacterial cells are also within the scope of the invention. Also included are certain molecular biology reaction mixtures that contain both double-stranded and single-stranded nucleic acids.

In its broadest aspects, the invention encompasses methods for separation and purification of double-stranded and single-stranded nucleic acids from the same sample, wherein double-stranded nucleic acid is selectively adsorbed to a mineral support in the presence of high concentration chaotropic salt at neutral pH. This is followed by a pH adjustment of the flow-through so that single-stranded nucleic acid is adsorbed to a second mineral support. Alternatively the flow-through can be mixed with a lower aliphatic alcohol followed by loading to and purification from a second column. As an example, the double-stranded nucleic acid can be genomic DNA, while the single-stranded nucleic acid can be RNA such as total RNA.

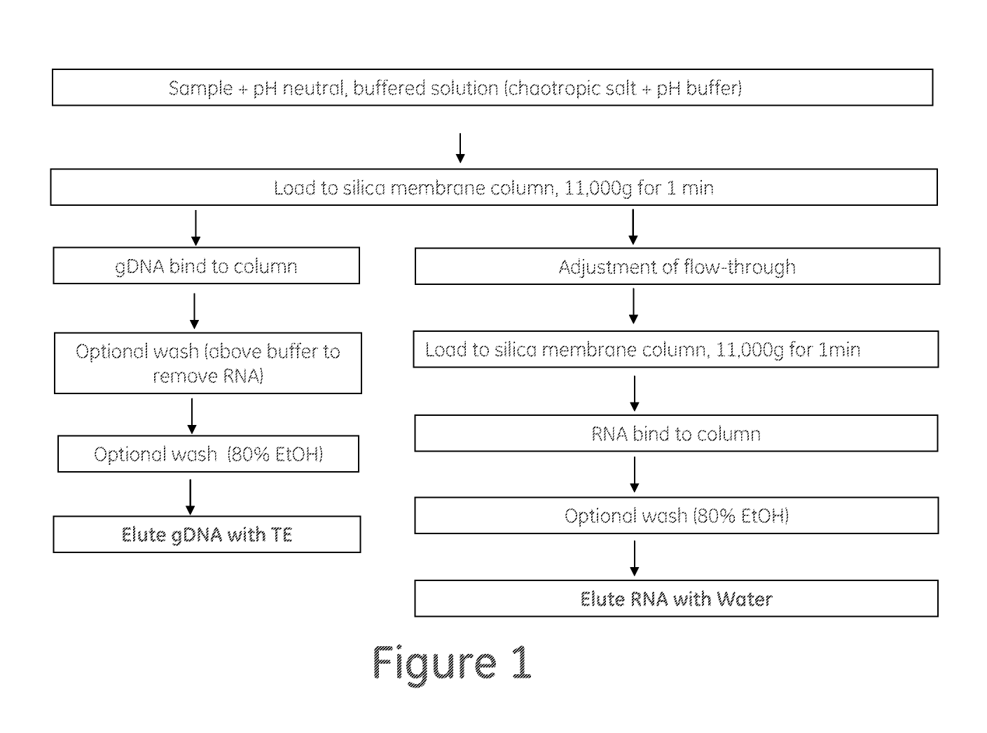

Accordingly, a sample solution containing both the double-stranded and single-stranded nucleic acids is admixed first with a pH-neutral, buffered solution containing a suitable chaotropic salt. The mixture is applied to a first mineral support under conditions for the double-stranded nucleic acid (e.g., genomic DNA) to bind; while the flow-through containing unbound single-stranded nucleic acids (e.g., total RNA) is collected. The follow-through is applied to a second mineral support under conditions for single-stranded nucleic acids to bind (e.g., by lowering the pH or mixing with a lower aliphatic alcohol) double-stranded and single-stranded nucleic acids are eluted from the first and second mineral support, respectively. An example workflow according to an embodiment of the invention is presented in FIG. 1.

The term “chaotrope” or “chaotropic salt,” as used herein, refers to a substance that causes disorder in a protein or nucleic acid by, for example, but not limited to, altering the secondary, tertiary, or quaternary structure of a protein or a nucleic acid while leaving the primary structure intact. Exemplary chaotropes include, but are not limited to, guanidine hydrochloride, guanidinium thiocyanate, sodium thiocyanate, sodium iodide, sodium perchlorate, and urea. A typical anionic chaotropic series, shown in order of decreasing chaotropic strength, includes: CCl₃COO⁻→CNS⁻→CF₃COO⁻→ClO₄⁻>I⁻→CH₃COO⁻→Br⁻, Cl⁻, or CHO₂⁻.

The term “lower aliphatic alcohol” as used herein is intended to refer to C₁-C₃ alcohols such as methanol, ethanol, n-propanol, or isopropanol. Preferably, the lower aliphatic alcohol is ethanol or an aqueous ethanol mixture.

One of the most important aspects in the purification of RNA is to prevent its degradation during the procedure. Therefore, the current solutions for separating the double-stranded and single-stranded nucleic acids are preferably solutions containing large amounts of chaotropic ions. This solution immediately inactivates virtually all enzymes, preventing the enzymatic degradation of RNA. The solution contains chaotropic substances in concentrations of from 0.1 to 10 M. As said chaotropic substances, there may be used, in particular, salts, such as sodium perchlorate, guanidinium chloride, guanidinium isothiocyanate/guanidinium thiocyanate, sodium iodide, potassium iodide, and/or combinations thereof.

Optionally, the solution also includes a reducing agent which facilitates denaturization of RNase by the chaotropes and aids in the isolation of undegraded RNA. Preferably, the reducing agent is 2-aminoethanethiol, tris-carboxyethylphosphine (TCEP), or beta-mercaptoethanol.

The solution used in the present invention preferably also contains a sufficient amount of buffer to maintain the pH of the solution. The pH should be maintained in the neutral range of about 6-8. The preferred buffers for use in the solution include tris(hydroxymethyl)aminomethane hydrochloride (Tris-HCl), sodium phosphate, sodium acetate, sodium tetraborate-boric acid and glycine-sodium hydroxide.

In a most preferred embodiment, a pH-neutral, buffered solution includes 7 M guanidine HCl, 50 mM Tris, at pH 7.

When the sample is a biological sample or cells, it is first lysed in an aqueous lysis system containing chaotropic substances and/or other salts by, in the simplest case, adding it to the cells. Some biological sample or cells cannot be lysed directly in aqueous systems containing chaotropic substances, such as bacteria, for instance, due to the condition of their cell walls. These starting materials must be pretreated, for example, with lytic enzymes, prior to being used in the process according to the invention. Preferably, the lysis solution also includes a reducing agent which facilitates denaturization of RNase by the chaotropes and aids in the isolation of undegraded RNA.

Optionally, the lysis solution also includes a non-ionic surfactant (i.e., detergent). Exemplary nonionic surfactants include, but are not limited to, t-octylphenoxy-polyethoxyethanol (TRITON® X-100), (octylphenoxy)polyethoxyethanol (IGEPAL® CA-630/NP-40), triethyleneglycol monolauryl ether (BRIJ® 30), sorbitari monolaurate (SPAN® 20), or the polysorbate family of chemicals. The polysorbate family of chemicals, such as TWEEN™ 20, is a preferred group of non-ionic detergents. Other commercially available polysorbates include TWEEN™ 40, TWEEN™ 60 and TWEEN™ 80 (Sigma-Aldrich, St. Louis, Mo.). Any of these and other related chemicals is effective as a replacement of TWEEN™ 20.

In a most preferred embodiment, the lysis solution includes 7 M guanidine HCl, 2% TWEEN™ 20 and 1% β-mercaptoethanol.

The lysis solution of the present invention preferably also contains a sufficient amount of buffer to maintain the pH of the solution.

Once the sample is admixed with a pH-neutral, buffered solution containing a suitable chaotropic salt, the mixture is applied to a mineral support. It is discovered that under pH neutral conditions, only the double-stranded nucleic acid (e.g., genomic DNA) binds to the mineral support, while the single-stranded nucleic acid (e.g., total RNA) does not bind and is collected as a flow-through by a simple spin.

The mineral support preferably includes porous or non-porous metal oxides or mixed metal oxides, silica gel, silica membrane, materials predominantly consisting of glass, such as unmodified glass particles, powdered glass, quartz, alumina, zeolites, titanium dioxide, zirconium dioxide. The particle size of the mineral support material ranges from 0.1 μm to 1000 μm, and the pore size from 2 to 1000 μm. Said porous or non-porous support material may be present in the form of loose packings or may be embodied in the form of filter layers made of glass, quartz or ceramics, and/or a membrane in which silica gel is arranged, and/or particles or fibers made of mineral supports and fabrics of quartz or glass wool, as well as latex particles with or without functional groups, or frit materials made of polyethylene, polypropylene, polyvinylidene fluoride, especially ultra high molecular weight polyethylene, high density polyethylene.

The flow-through from the first mineral support contains single-stranded nucleic acid (e.g., total RNA). The flow-through is acidified and applied to a second mineral support. It is discovered that at lower, acidic pH, single-stranded nucleic acid (e.g., RNA) binds to the mineral support. A simple centrifugation step separates the single-stranded nucleic acid bound to the mineral support from the flow-through.

As an example, a suitable pH for the single-stranded nucleic acid to bind is a pH of between 2-6, such as between pH 3-6, or more preferably between pH 4-6. The most suitable pH for the single-stranded nucleic acid to bind is around pH 5. There are many methods to lower the pH of the flow-through. In one embodiment, acetic acid is used to achieve a lowered pH for the flow-through, prior to loading onto the second mineral support.

Alternatively the flow-through can be mixed with a lower aliphatic alcohol such as 70% ethanol, followed by loading to and purification from the second column. In addition, the flow-through can be recovered by a variety of other methods available. This includes alcohol precipitation or other chromatography separation methods.

The second mineral support for single-stranded nucleic acid binding includes a similar material as the first mineral support described above. Preferably, the first mineral support and the second mineral support are each silica membranes.

The double-stranded nucleic acid adsorbed on the first mineral support and the single-stranded nucleic acid adsorbed on the second mineral support can be eluted under conditions of low ionic strength or with water, respectively.

Optionally, wash steps may be performed prior to the elution of the respective nucleic acid (single-stranded nucleic acid or double-stranded nucleic acid). For purifying the double-stranded genomic DNA, an optional wash of the first mineral support (i.e., column) can be applied to removes any residual single-stranded nucleic acid. This wash is performed with a solution similar in composition to the pH-neutral, buffered binding solution containing chaotropic salt. Further, a wash buffer containing a high concentration of organic solvents such as lower aliphatic alcohols, can be used to wash the first and second mineral support prior to elution of the desired nucleic acid, to remove components other than the bound nucleic acids.

Following the work flow illustrated in FIG. 1 and the experimental conditions as further described in the Examples below, double-stranded and single-stranded nucleic acids have been successfully purified from sample mixtures, with a high recovery rate.

Also provided is a kit for the separation and/or purification of double-stranded and single-stranded nucleic acids from a sample. The kit comprises: a pH-neutral, buffered solution consisting essentially of a chaotropic salt and a pH buffer for sample processing; a first mineral support for binding the double-stranded nucleic acid; a second mineral support for binding the single-stranded nucleic acid; an elution solution for eluting double-stranded nucleic acid from the first mineral support; and an elution solution for eluting single-stranded nucleic acid from the second mineral support.

Preferably, the pH-neutral, buffered solution in the kit includes a chaotropic salt and a pH buffer. Most preferably, the lysis solution includes 7 M guanidine HCl, 50 mM Tris, pH 7.

The mineral support may be present in loose packing, fixed between two means (for example frits, plugs made out of a suitable inert material, etc), or in the form of membranes which are arranged within the hollow body of a column. Preferably, the first mineral support and the second mineral support are each silica membranes.

Other features and advantages of the invention will be apparent from the following examples and from the claims.

### EXAMPLES

The following examples serve to illustrate the process for the separation and purification of double-stranded and single-stranded nucleic acids from a sample according to embodiments of the present invention and are not intended to be limiting.

### Solutions and Protocols

### 1. Solutions and Columns Used in the Examples

| Description | Composition |
|---|---|
| pH-neutral, buffered | 7M Guanidine HCl, 50 mM Tris, pH 7 |
| solution | |
| Wash buffer | 10 mM Tris, 1 mM EDTA, pH 8 (before use, 4 |
| | parts of ethanol added to 1 part of buffer) |
| DNA elution buffer | 10 mM Tris, 0.5 mM EDTA, pH 8 |
| RNA elution buffer | Water |
| ILLUSTRA ™ | silica membrane spin column |
| genomicPrep tissue | |
| and cell mini column | |

### 2. Double-Stranded Nucleic Acid Purification

### 2.1 Double-Stranded Nucleic Acid Binding

a. Place a new spin column into a new collection tube.
b. Transfer ˜350 μl mixture of the sample containing both double-stranded and single-stranded nucleic acid and the pH neutral, buffered solution to the column.
c. Centrifuge at 11,000×g for 1 min.
d. Save the flow-through for purification of single-stranded nucleic acid.
e. Transfer the column to a new 2 ml collection tube.

### 2.2 Optional Column Wash

a. Add 500 μl of the pH-neutral, buffered solution to the column.
b. Centrifuge at 11,000×g for 1 min. Discard the flow through.
c. Place the column back into the same collection tube.
d. Add 500 μl of Wash buffer to the column.
e. Centrifuge at 11,000×g for 1 min.
f. Transfer the column to a clean 1.5 ml microcentrifuge tube.

### 2.3 Double-Stranded Nucleic Acid Elution

a. Add 100 μl Elution buffer to the center of the column.
b. Centrifuge at 8,000×g for 1 minute.
c. Discard the column and store the tube containing pure double-stranded nucleic acid at −20° C.

### 3. Single-Stranded Nucleic Acid Purification

### 3.1 Single-Stranded Nucleic Acid Binding

a. Place a new spin column in a new collection tube.
b. Add 1.75 μl of acetic acid to the flow-through from step 2.1.d. Mix well by pipetting up and down several times. Transfer the entire mixture to the column.
c. Centrifuge at 11,000×g for 1 min.
d. Discard the flow-through.
e. Transfer the column to a new 2 ml collection tube.

### 3.2 Optional Column Wash

a. Add 500 μl of Wash Buffer to the column.
b. Centrifuge at 11,000×g for 1 min.
c. Transfer the column to a clean 1.5 ml microcentrifuge tube.

### 3.3 Single-Stranded Nucleic Acid Elution

a. Add 100 μl of Elution buffer to the center of the column.
b. Centrifuge at 8,000×g for 1 minute.
c. Discard the column and store the tube containing single-stranded nucleic acid at −20° C. (DNA) or −80° C. (RNA) until needed.

### Example 1

### Separation and Purification of a Sample Containing Both Genomic DNA and Total RNA

To find an optimal protocol for the separation (and purification) of double-stranded and single-stranded nucleic acids, we experimented with a mixture of purified genomic DNA and purified RNA. Effective separation was achieved using the following method.

We mixed 2 μg of purified rat liver genomic DNA and 2 μg of purified rat liver total RNA with a variety of different buffer solutions and followed the protocol above for double-stranded nucleic acid purification. We then adjusted the pH of the flow-through to various acidic pH and followed the protocol above for single-stranded nucleic acid purification. We found that a pH-neutral, buffered solution with a working composition of 7 M Guanidine HCl, 50 mM Tris, pH 7 is optimal for selective binding of DNA to the first column. We also found that a slight reduction in pH is effective for RNA binding to the second column. A simple pH adjustment of the flow through from 7 to 5 (e.g., with acetic acid) allows RNA to bind to the second column (data not shown).

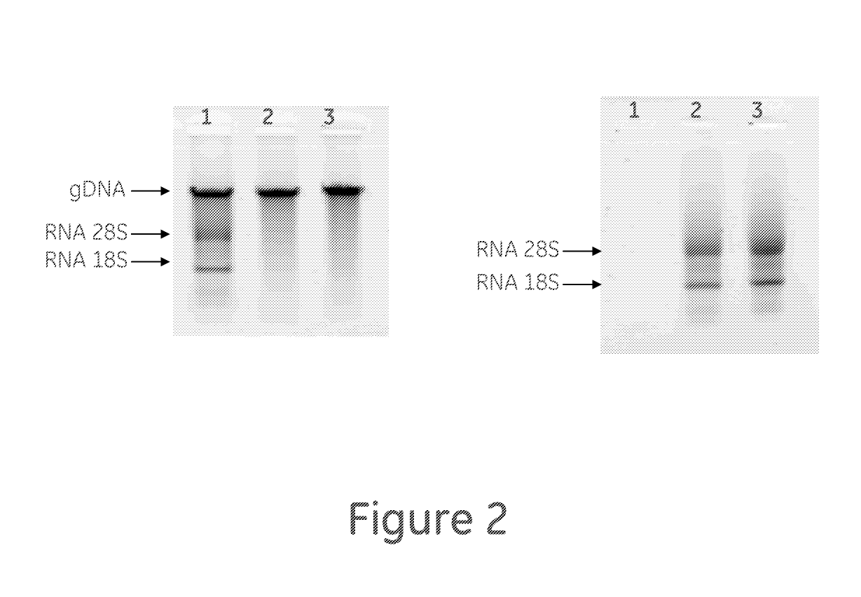

FIG. 2 presents data for three representative experiments of genomic DNA and total RNA separation, showing successful separation using the optimized process. The starting material each contained 2 μg of purified genomic DNA and 2 μg of purified total RNA. The above protocols were followed unless noted below. The elution was performed in 100 μl volume and 15 μl was loaded for each well. In the first experiment, the pH of the pH neutral, buffered solution (350 μl of 7 M Guanidine HCl, 50 mM Tris, pH 7) was adjusted to pH 5 prior to mixing with the DNA and RNA sample. The flow through from the first column was mixed with 0.7 volume of alcohol prior to loading to the second column. Both RNA and DNA became bound to the first column at pH 5, and was eluted there from, leaving nothing in the flow through to binding to (and elute from) the second column (See Lane 1 of each gel panel).

In the second experiment, the pH of the pH-neutral, buffered solution (350 μl of 7 M Guanidine HCl, 50 mM Tris, pH 7) was kept at pH 7 prior to mixing with the DNA and RNA sample. The flow-through from the first column was mixed with 1.75 μl of acetic acid to adjust the pH to about 5, prior to loading of the second column. Only DNA became bound to the first column at pH 7, and was eluted therefrom. RNA bound to the second column was eluted there from (See Lane 2 of each gel panel).

In the third experiment, the pH of the pH-neutral, buffered solution (350 μl of 7 M Guanidine HCl, 50 mM Tris, pH 7) was kept at pH 7 prior to mixing with the DNA and RNA sample. The flow-through from the first column was mixed with 0.7 volume of alcohol prior to loading to the second column. As expected, only DNA became bound to the first column at pH 7, and was eluted there from. RNA became bound to the second column as expected and was eluted there from (See Lane 3 of each gel panel).

The experiments demonstrate that at pH 7, genomic DNA in a solution containing chaotropic salt would bind to the silica membrane column, but RNA will not bind. However, at pH 5, both will bind to the column. A simple adjustment of the pH of the flow-through from pH 7 to 5 enables RNA binding to the column. This pH adjustment achieves similar effect for RNA binding as the use of a lower aliphatic alcohol.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for the separation of double-stranded nucleic acid from single-stranded nucleic acids, which method comprising:
a) mixing a sample containing said double-stranded nucleic acid and said single-stranded nucleic acid with a pH-neutral, buffered solution consisting essentially of a chaotropic salt and a pH buffer to generate a mixture;
b) applying said mixture to a first mineral support for the double-stranded nucleic acid to bind; and
c) collecting the flow-through which contains unbound single-stranded nucleic acid.

2. The method of claim 1, further comprising recovering the double-stranded nucleic acid from the first mineral support.

3. The method of claim 2, further comprising optionally wash the first mineral support prior to the recovery of the double-stranded nucleic acid.

4. The method of claim 1, further comprising:
d) adjusting the pH of said flow-through to an acidic pH;
e) applying said acidified flow-through from step (d) to a second mineral support for the single-stranded nucleic acid to bind.

5. The method of claim 4, further comprising recovering the single-stranded nucleic acid from the second mineral support.

6. The method of claim 5, further comprising optionally wash the second mineral support prior to the recovery of the single-stranded nucleic acid.

7. The method of claim 1, wherein said double-stranded nucleic acid is double-stranded DNA and said single-stranded nucleic acid is RNA.

8. The method of claim 1, wherein said sample is a lysate of cultured cells, microorganisms, plant or animal cells.

9. The method of claim 1, wherein said pH-neutral, buffered solution has a pH between about 6 and 8.

10. The method of claim 1, wherein said chaotropic salt is guanidine HCl.

11. The method of claim 1, wherein said pH-neutral, buffered solution has a pH of about 7.0 and consisting essentially of 7 M guanidine HCl, 50 mM Tris-HCl.

12. The method of claim 4, wherein the first mineral support and the second mineral support are porous or non-porous and comprised of metal oxides or mixed metal oxides, silica gel, silica membrane, glass particles, powdered glass, quartz, alumina, zeolite, titanium dioxide, or zirconium dioxide.

13. The method of claim 12, wherein the first mineral support and the second mineral support are each silica membranes.

14. The method of claim 4, wherein said acidic pH is between about 2 and 6.

15. The method of claim 4, wherein said acidic pH is about 5.

16-19. (canceled)
